(19) United States
(12) Patent Application Publication
(10) Pub. No.: US 2001/0039325 A1
(43) Pub. Date: Nov. 8, 2001

(54) HYDROXYAROMATIC-MASKED ISOCYANATES

(76) Inventors: PIERRE ARDAUD, SAINTE-FOY-LES- (FR); JEAN-MARIE BERNARD, MORNANT (FR)

Correspondence Address: BURNS DOANE SWECKER & MATHIS L L P, POST OFFICE BOX 1404, ALEXANDRIA, VA, 22313-1404, US

(21) Appl. No.: 08/960,620

(22) Filed: Oct. 29, 1997

Related U.S. Application Data
(63) Continuation of application No. 08/434,535, filed on May 4, 1995, now abandoned.

(30) Foreign Application Priority Data
May 4, 1994 (FR) 94 05436

(51) Int. Cl. C08G 18/80; C07C 271/06

(52) U.S. Cl. 528/45; 525/424; 525/454; 528/49; 528/55; 528/58; 528/73; 528/80; 528/85; 560/24; 560/25; 560/26; 560/115; 560/157; 560/158; 560/330; 560/335; 560/336; 560/355; 560/358

(57) ABSTRACT

Unique masked isocyanates well suited for formulation into a variety of coating compositions, for example paint powders, are prepared by condensing an isocyanate with a ring-hydroxylated aromatic compound bearing at least one substituent which comprises a carbonyl or nitrile functional group and the apparent melting point of which being at least 30° C.

### BACKGROUND OP THE INVENTION

1. Technical Field of the Invention

The present invention relates to a novel class of masked isocyanates, and, more especially, relates to isocyanates masked by means of hydroxyaromatic compounds and to the use of such masked isocyanates in powder coating techniques.

2. Description of the Prior Art

For reasons associated with environmental protection and safety in the workplace, it is increasingly important to eliminate the use of solvents in coating techniques, and especially in paint techniques.

In this context, coating techniques using powders for example, electrostatic coating, are being increasingly widely used.

Masked isocyanates find application in this art, but their use is limited by the few compounds satisfying the chemical requirements of the powders.

One disadvantage is presented by the scarcity of masked isocyanates or mixtures of isocyanates which remain in powder form under the usual storage conditions which may vary greatly from one location to another. This requires that such compounds have a relatively high melting point and/or glass transition temperature (Tg).

The masked isocyanates do not always have a sharp melting point, and in this case an apparent melting point is therefore determined, either with a koffler block or via a technique of capillary type (for example the so-called “Büchi” melting point). Class transition temperature may be measured by differential thermal analysis (DTA) techniques.

These compounds should also have glass transition temperatures and melting points which are sufficiently low to permit them to react in the conditions under which powders are used.

In addition, the compounds derived via crosslinking reactions should not be harmful or toxic, either to the health of humans or animals, or to the environment.

### SUMMARY OF THE INVENTION

Accordingly, a major object of the present invention is the provision of a novel class of masked or blocked isocyanates which satisfies the aforesaid criteria.

Another object of the present invention is the provision of novel coating compositions which are useful in powder coating techniques and which contain blocked isocyanates.

Yet another object of this invention is the provision of a process for the synthesis of the isocyanates satisfying the above criteria.

Briefly, the present invention features novel masked isocyanates, whether pure or in admixture, which are prepared by the condensation of an aromatic compound which is hydroxylated on the ring member and bears a functional group comprising nitrile functions or, preferably, carbonyl functions, with an isocyanate.

### DETAILED DESCRIPTION OF BEST MODE AND PREFERRED EMBODIMENTS OF THE INVENTION

More particularly according to the present invention, among the subject masked isocyanates, those for which it is possible to determine an apparent melting point are advantageously selected, this measurement being made at room temperature (20° C.). This melting point should be at least equal to 30° C. (one significant figure) and advantageously at least 50° C.

It is desirable for the compounds not to cake or lump together; thus, compounds which, when ground and stored at room temperature, have a similar particle size after a 24-hour interval, are selected.

The lumping characteristic is generally more or less associated with the glass transition temperature (Tg); hence, the preferred compounds are those which have a glass transition temperature (Tg) at least equal to 10° C. (two significant figures), advantageously at least 20° C. (one, preferably two, significant figures) and preferably at least 30° C. (two significant figures).

The selection of alkyl substituents or moieties may be important, especially in the case of the alkyl hydroxybenzoates, more specifically for the para-hydroxybenzoate. Thus, the esters whose alkyl moiety is linear and contains more than two carbons either have an insufficiently high melting point or are syrupy and crystallize only after a long period of time ranging from one week to several months, which makes them difficult to use, and they are thus not preferred. Hence, the n-propyl, n-butyl and more generally n-alkyl esters are difficult to use. In addition, long chains should also be avoided for similar reasons, especially those in which the number of carbons is greater than six.

The ethyl radical is an intermediate case and provides results which are acceptable (but only when its content in the starting material is low, below 2% and preferably below 1% in total mass), but not excellent. The isopropyl radical and, especially, the methyl radical, are the preferred.

The nitrile and, preferably, the carbonyl functional groups may be attached to the ring member either by a single valence bond or via a linking group which may be a chalcogen, nitrogen or phosphorus bearing a hydrogen atom or a substituent, or an optionally substituted methylene bridge.

Given that the electron-withdrawing influence decreases or disappears on insertion of a linking group or bridge between the electron-withdrawing group and the ring member, direct bonding between the ring and the electron-withdrawing group is preferred, provided there does not already exist an electron-withdrawing group on the ring, or that this group is not naturally electron-poor (six-membered heterocycle for example).

Chalcogen linking groups or linking groups only substituted by hydrogen atoms or, and to a lesser extent, methyl radicals, are preferred; linking groups based on element(s) from the second row (the row containing oxygen) of the Periodic Table are also preferred.

According to the present invention, the masked isocyanate, whether pure or in admixture, is characteristically prepared from a polyisocyanate, namely, one possessing at least two isocyanate functions, advantageously more than two (possibility of fractional values since these are generally mixtures of more or less condensed oligomers), which is itself typically prepared via precondensation or via a prepolymerization of a unitary diisocyanate (or elemental diisocyanate, i.e., the isocyanate functions borne thereby not having been subjected to condensation(s) with another isocyanate function (in the case of biuret) or polymerization(s) (in the case of dimers or trimers, especially of those contributing to the isocyanuric ring)). Exemplary elemental isocyanates include those comprising a hydrocarbon molecular skeleton bearing at least two isocyanate functional groups. Such a skeleton, or backbone, is typically an arylene radical, an alkylene radical (including aralkylene), for example a polymethylene backbone (notably hexamethylene), or that required to constitute IPDI. Also, such skeletons may comprise alkyl moieties at one end thereof and aryl moieties at the other. The atomic weight of these elemental isocyanates is advantageously at most 300 (one significant figure), preferably at most 200 (one significant figure).

In general, the average molecular weights of these prepolymers or of these precondensates is not more than 2,000 (one significant figure), usually not more than 1,000 (one significant figure, preferably two).

Thus, among the suitable polyisocyanates according to this invention, exemplary are those of the biuret type and those for which the di- or trimerization reaction has created four-, five- or six-membered rings. Among the six-membered rings, representative are the isocyanuric rings derived from a homo- or hetero-trimerization of various diisocyanates alone, with other isocyanate(s) (mono-, di- or polyisocyanate(s)), or with carbon dioxide, and in this case a nitrogen atom of the isocyanuric ring is replaced by an oxygen atom.

The preferred polyisocyanates are those which have at least one aliphatic isocyanate function, namely, at least one isocyanate function masked according to the invention is attached to the molecular skeleton via an sp³-type carbon advantageously bearing a hydrogen atom, preferably two.

The aromatic compound hydroxylated on the ring member, which serves to mask the isocyanate function, is advantageously selected from among those of formula (I):

Ar(R)ₙ(Y-Z)ₘ(OH)ₚ (I)

in which Ar is an aromatic nucleus substituted by n substituents R, m polar functional groups Z which are nitrile or carbonyl groups, and p hydroxyl functions.

The values of n, m and p are positive integers or zero and are such that the sum n+m+p is not more than the number of substitutable ring positions; p is advantageously not more than 2, and is preferably equal to 1.

m is advantageously not more than two, and is preferably equal to 1.

n is advantageously not more than 3, preferably is zero, one and two, and more preferably is equal to zero.

R represents substituents which are immaterial and inert with respect to the masking reaction and generally are hydrocarbon radicals, typically alkyl radicals in the stymological sense of the term, namely, an alcohol whose hydroxyl function has been removed.

Two vicinal substituents R may together form a ring member, which may, for example, be aromatic.

Z is advantageously a moiety bearing a carbonyl function. Exemplary of these are alkoxycarbonyl functional groups (i.e., ester functions), the amide function, the ketone function with the preferred condition that there exist no acidic hydrogen (namely, the function advantageously does not bear hydrogen substituents or, if it does, the corresponding pKa is at least equal to about 20 (one significant figure, preferably two) and is more preferably at least equal to about 25) in a position α-to the carbonyl function (ester, ketone or amide). Thus, the preferred amides (including lactam or even urea) are advantageously substituted, preferably sufficiently that there exist no hydrogen atoms on the nitrogen of the amide function, or such that no reactive hydrogen atom is present.

Y is a divalent bridge, advantageously —O—, —S—, —NR′— or —CR′R″—, wherein R′ and R″ are hydrogen atoms or hydrocarbon radicals, advantageously alkyl radicals having from 1 to 6 carbon atoms, preferably having from 1 to 4 carbon atoms, preferably methyl, more preferably hydrogen.

Y is preferably a single valence bond.

It is preferable for the polar function or functions z (in general the nitrile function and/or the carbonyl functions) not to be vicinal to the group Z as, for example, in salicylic acid.

The aromatic nucleus Ar comprises one or more ring members, which are advantageously condensed hetero- or homocyclic rings. It is preferable for Ar not to contain more than two rings, and preferably not more than one ring.

The aromatic nucleus Ar may comprise one or more hetero- or homocyclic rings, typically homocyclic ring because of their ready accessability. However, the advantage presented by 6-membered heterocycles, which have a very much lower release temperature than that of the corresponding homocycles, should be emphasized.

It is desirable that the total number of carbon atoms in the aromatic compound hydroxylated on the ring member be not more than 20, preferably not more than 10 (one significant figure).

This ring advantageously contains 6 members, the ring members being carbon or nitrogen with the required number of substituents to satisfy the valency of these atoms.

Among the acids whose derivatives provide the most satisfactory results, acids bonded to a benzene ring are exemplary. Thus, meta-hydroxy- and para-hydroxybenzoic acids, and especially the esters thereof, provide good results.

As indicated above, according to the present invention it is preferable for the melting point of the compound or of the compound mixture obtained to have an apparent melting point at least equal to 30° C., preferably at least 50° C.

It is also preferable for the glass transition temperature to be at least equal to 20° C., advantageously at least 40° C.

It is preferable to select compounds according to the present invention such that they react completely with a primary alcohol at 250° C. in less than half an hour. The reaction is considered to be complete if it is attained to a degree of 90% or more. The isocyanates for which the invention is most advantageous are those in which the nitrogen atom is attached to an sp³-hybridized carbon and more particularly to aliphatic isocyanates, and especially to polymethylene diisocyanates and the various condensation derivatives thereof (biuret, etc.) and di- and trimerization derivatives thereof.

According to the present invention, it is preferable and in certain instances necessary for the percentage of residual free isocyanate groups to be not more than 5%, advantageously not more than 3%, preferably not more than 1%. The highest melting points or glass transition temperatures are obtained with percentages not exceeding 0.5%. The contents of aromatic compound hydroxylated on the ring are also advantageously low, namely, not more than 5%, advantageously not more than 3%, preferably not more than 1%.

Also as indicated above, the present invention also features powder coating compositions which contain a masked polyisocyanate or a mixture of masked polyisocyanates.

As utilized herein, the particle size characteristics often make reference to notations of the type dₙ where n is a number ranging from 1 to 99; this notation is well known in many technical fields, but slightly rarer in chemistry; thus, its definition is as follows: This notation represents the particle size such that n % (in weight, or more exactly in mass, since weight is not an amount of material but a force) of the particles is less than or equal to the said size.

In the powder compositions according to the present invention, the subject masked isocyanates advantageously constitute a population (which is advantageously distinct from that of the coreactants) of particles whose d₉₀ is not more than 200 microns, advantageously not more than 100 microns, preferably not more than 50 microns; this particle population has a d₁₀ at least equal to 1 micron, advantageously to at least 5 microns, preferably to at least 10 microns.

The powder compositions advantageously contain at least one polyol (at least a diol) or, in certain instances, polyamines. It is also possible to include polyfunctional compounds having at least two functional groups selected from among amine functions or -ols (phenols or preferably alcohols) and the above compounds may additionally be substituted by other functional groups (for example an acid function such as a carboxylic or sulfonic acid function) on condition that these functional groups do not prevent the condensation or the crosslinking thereof.

These polyols or polyamines themselves are also in the form of powders and satisfy the same melting point and glass transition temperature criteria as those indicated above.

It is preferred that the melting point of the compositions according to the present invention be at least equal to 50° C., and it is even desirable for the softening temperature thereof to be such that there is no sintering of the powder at a temperature of at least 50° C.

It is also preferable for the glass transition temperature thereof to be at least equal to 40° C.

Advantageously, the powder compositions also contain at least one catalyst, generally and preferably curing catalysts based on tin or zinc.

Where appropriate, they contain additives and adjuvants which are conventional in this art, such as fillers, pigments (TiO₂, etc.) and additives for enhancing physical properties (surface tension, resistance to aging and light, ease of use, etc.).

According to the present invention, one preparative technique entails contacting the free, or partially free, isocyanate with the hydroxyaromatic compound, namely, the compound of phenol type, in a solvent.

When the compounds according to the invention and the precursors thereof are stable under the conditions indicated below, the synthesis may be carried out without solvent, but in the molten state. The final product is then cooled, for example via the flaking thereof, which may be attained by abrupt cooling effected by pouring the reaction mixture onto a cold wall surface. The flakes obtained may be ground. In order to obtain good (that is to say, low) percentages of residual free isocyanate function, it is important to introduce the aromatic compound hydroxylated on the ring nucleus in an amount very close to the stoichiometric amount. It is preferable to be in a slight stoichiometric excess (of 0.5% to 2%, preferably not more than 1%).

It is also preferable to add a catalyst for the condensation of the isocyanates to -ol functions; these condensation catalysts are typically based on tin or tertiary amine.

The temperature at the end of the condensation reaction is advantageously not more than 100° C. (one significant figure, preferably two), preferably not more than 80° C. and advantageously at least equal to 50° C., preferably to 60° C. Indeed, if overheated, the risk exists of the percentage of free isocyanate functions being too high.

When a solvent is present, it is preferably selected such as to be sufficiently polar to dissolve at least 50, preferably at least 100 and more preferably at least 200 grams per liter of initial isocyanate.

Once the reaction is complete, the final product should be precipitated, according to a per se standard crystallization technique and more preferably by addition of a precipitating compound which is sufficiently nonpolar to effect the precipitation without there necessarily being any crystallization.

The precipitating compound is, of course, a compound of volatile type and usually compounds of light hydrocarbon mixture family of the petroleum ether type, or of the hexane or heptane type. It is also possible to use, whether alone or in admixture, ethers of light alcohols (namely, alcohols containing not more than six carbon atoms, preferably not more than 4).

Compounds of the alkane or alkene type in which the number of carbons is less than 20 and greater than 4 are advantageously employed.

In order to further illustrate the present invention and the advantages thereof, the following specific examples are given, it being understood that same are intended only as illustrative and in nowise limitative.

### EXAMPLE 1

### Masking of Tolonate HDT by Condensation with Methyl p-hydroxybenzoate

The following materials were charged into a 500 ml reactor:

(i) hexamethylene diisocyanate trimer marketed under the trademark Tolonate HDT®=54.2 g (NCO number=22.1%); and

(ii) Solvesso 100®=25 g.

The following was then added in several fractions, with stirring and at room temperature:

(iii) methyl p-hydroxybenzoate=47.6 g (0.31 mol).

The reaction mass was heated to 60° C. and maintained at this temperature until the NCO functions had disappeared.

After cooling, the desired final product (blocked polyisocyanate) precipitated. It was converted into a powder and washed with n-hexane:

n-hexane=41.2 g.

The reaction mass was filtered and the solid obtained was washed with several fractions of hexane and then ground and again dried.

(a) weight obtained=95.7 g

(b) melting point=85° C.

peaks were observed via NMR:

(1) 7.8 ppm (hydrogen borne by the nitrogen of the carbamate;

(2) 7.9 ppm (aromatic hydrogen ortho- to the carbamic ester function);

(3) 7.10 ppm (aromatic hydrogen ortho- to the carbonyl function).

### EXAMPLE 2

### Glaze Formulation of the Masked Isocyanate of Example 1

The masked isocyanate (i) obtained in Example 1 was formulated in the following manner:

(i) I=6.0 g;

(ii) Desmophen 690®=14.0 g (% OH=2%), i.e., an NCO/OH ratio=1.

The mixture of the two powders was ground until a perfectly homogenous mixture having a particle size of less than 50 μm was obtained.

Some of this powder was applied as a layer 300 μm thick onto a steel plate and was heat-treated at various temperatures for 30 or 60 minutes, as reported in the Table I which follows:

TABLE I

| | 30 minutes | | 60 minutes | |
|---|---|---|---|---|
| | Test | Hardness | Test | Hardness |
| | Solvent | while hot | Solvent | while hot |
| CURING | (2) | (1) | (2) | (1) |
| 130° C. | D | M | D | M |
| 160° C. | D | M | D | M |
| 190° C. | D | M | I | VG |
| 200° C. | I | VG | I | VG |

The film obtained was qualified by its hardness and its solvent-resistance:

(1) VG=very good: M=mediocre,

(2) Deposition of a drop of methyl ethyl ketone and observation of the deterioration of the film,

D=the film was degraded by the action of solvent,

I=the film was intact after the action of solvent.

### EXAMPLE 3

The following material was introduced into a reactor:

(i) Tolonate HDTO: 100 g (0.529 mol NCO).

The following was added thereto:

(ii) Methyl p-hydroxybenzoate: 81.3 g (0.529 mol).

The mixture was heated and stirred until melting of the blocking agent was attained, at about 85° C.; at about 100° C. the medium was totally clear and colorless.

It was heated to 120° C. and maintained at this temperature for 1 hour.

After cooling, the product was in the form of a slightly sticky hard gum:

Tg=8° C.

By assaying with dibutylamine, the content of free NCO functions was determined to be about 10%.

### EXAMPLE 4

The following material was introduced into a reactor:

(i) Tolonate HDT®: 100 g (0.529 mol NCO).

The following was added thereto:

(ii) Methyl p-hydroxybenzoate: 81.3 g (0.529 mol), and

(iii) Triethylamine (TEA): 0.2 g

The mixture was heated and stirred until melting of the blocking agent was attained, at about 85° C.; at about 100° C. the medium was totally clear and colorless.

After maintaining the mix at 100° C., the content of free NCO functions was measured by assaying using dibutylamine:

1 h at 100° C.: free NCO=6.2%

2 h at 100° C.: free NCO=5.6%

5 h at 100° C.: free NCO=5.6%

### EXAMPLE 5

The following material was introduced into a reactor:

(i) Tolonate HDT®: 100 g (0.529 mol NCO).

The following were added thereto:

(ii) Methyl p-hydroxybenzoate: 81.3 g (0.529 mol), and

(iii) Triethylamine (TEA): 0.2 g.

The mixture was heated and stirred until melting of the blocking agent was attained, at about 85° C.; at about 100° C. the medium was totally clear and colorless.

The reaction mass was then cooled gradually to 60° C. and then maintained at this temperature.

The content of free NCO functions was measured by assaying with dibutylamine:

| | |
|---|---|
| 30 min at 60° C.: | free NCO = 3.1% |
| 1 hr at 60° C.: | free NCO = 2.6% |
| 4 h at 60° C.: | free NCO = 2.6% |

### EXAMPLE 6

The following reagent was introduced into a reactor:

(i) Tolonate HDT®: 100 g (0.529 mol NCO).

The following were added thereto:

(ii) Methyl p-hydroxybenzoate: 81.3 g (0.529 mol), and

(iii) Triethylamine (TEA): 0.5 g.

The mixture was heated and stirred until melting of the blocking agent was attained, at about 85° C.; at about 100° C. the medium was totally clear and colorless.

The reaction mass was then cooled gradually to 60° C. and then maintained at this temperature.

The content of free NCO functions was measured by assaying with dibutylamine:

3 h at 60° C.: free NCO=1.2%

An additional 0.5 g of TEA was added to the reaction medium and, after again maintaining the temperature, the free NCO functions were then assayed:

3 h at 60° C.: free NCO 0.2%

After cooling, the product was in the form of a hard gum which may be ground:

Tg=24° C.

### EXAMPLE 7

The procedure of Example 4 was repeated, except that instead of the methyl p-hydroxybenzoate, the following compound was used:

ethyl p-hydroxybenzoate: 88.9 g (0.529 mol)

After cooling, the product was in the form of a solid gum:

Tg=18.8° C.

### EXAMPLE 8

The procedure of Example 4 was repeated, except that instead of the methyl p-hydroxybenzoate, the following compound was used:

butyl p-hydroxybenzoate: 77.8 g (0.529 mol)

After cooling, the product was in the form of a vitreous liquid:

Tg=5.5° C.

### EXAMPLE 9

The procedure of Example 4 was repeated, except that instead of the methyl p-hydroxybenzoate, the following compound was used:

isopropyl p-hydroxybenzoate: 73.1 g (0.529 mol)

After cooling, the product was in the form of a solid gum:

Tg=23° C.

### EXAMPLE 10

### Glaze Formulation of the Masked Isocyanate of Example 1

The masked isocyanate (i) obtained in Example 1, methyl p-hydroxybenzoate blocked with Tolonate HDT, was formulated in the following manner:

I=38.3 g

Johnson 587®=61.7 g (% OH=2.8%), i.e., an NCO/OH ratio=1.1.

The mixture of the two powders was ground until a perfectly homogeneous mixture having a particle size of less than 50 μm was obtained.

A fraction of this powder was applied as a layer 200 μm thick onto a steel plate and was heat-treated at various temperatures for 30 minutes.

The results obtained are reported in Table II below:

TABLE II

30 minutes

| | | Hardness |
|---|---|---|
| | Test Solvent | while hot |
| CURING | (2) | (1) |
| 130° C. | D | M |
| 14O° C. | D | M |
| 150° C. | D | M |
| 160° C. | I | VG |

The film obtained was qualified by its hardness and its solvent-resistance:

(1) VG=very good: M=mediocre

(2) Deposition of a drop of methyl ethyl ketone and observation of the deterioration of the film,

D=the film was degraded by the action of solvent,

I=the film was intact after the action of solvent.

While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A masked isocyanate comprising the condensate of an isocyanate with a ring-hydroxylated aromatic compound bearing at least one substituent which comprises a carbonyl or nitrile functional group and the apparent melting point of which being at least 30° C.

2. The masked isocyanate as defined by claim 1, said isocyanate comprising a polyisocyanate.

3. The masked isocyanate as defined by claim 2, said polyisocyanate comprising at least one elemental, at least partially aliphatic diisocyanate.

4. The masked is isocyanate as defined by claim 3, said polyisocyanate comprising a biuret or trimer compound of at least one elemental isocyanate.

5. The masked isocyanate as defined by claim 1, said hydroxylated aromatic compound having the formula (I):
Ar(R)ₙ(Y-Z)ₘ(OH)ₚ (I)
in which Ar is an aromatic nucleus; R is a hydrocarbon radical; Z comprises a nitrile or carbonyl functional group; Y is a divalent bridge; and m, n and p are each positive integers or zero such that the sum n+m+p is not greater than the number of substitutable ring positions of Ar.

6. The masked isocyanate as defined by claim 5, wherein formula (I), p is 1 and m is no greater than 2.

7. The masked isocyanate as defined by claim 5, wherein formula (I), Z comprises an (alkoxycarbonyl, amide, or alkylcarbonyl functional group, with the proviso that said alkylcarbonyl functional group contains no hydrogen atom on a carbon atom vicinal to the carbonyl function.

8. The masked isocyanate as defined by claim 5, wherein formula (I), no hydroxyl group is vicinal to a functional group Z.

9. The masked isocyanate as defined by claim 5, wherein formula (I), Ar is a carbonaceous or nitrogen-containing six-membered aromatic ring.

10. The masked isocyanate as defined by claim 1, said hydroxylated aromatic compound comprising a derivative of para- or meta-hydroxybenzoic acid.

11. The masked isocyanate as defined by claim 1, said hyroxylated aromatic compound comprising a para-hydroxybenzonitrile.

12. The masked isocyanate as defined by claim 1, having an apparent melting point of at least 30° C.

13. The masked isocyanate as defined by claim 1, having a glass transition temperature of at least 10° C.

14. The masked is isocyanate as defined by claim 1, at least one nitrogen atom of at least one isocyanate function of said isocyanate being bonded to an sp³-hybridized carbon atom.

15. A coating composition comprising a powder of at least one masked isocyanate as defined by claim 1.

16. The coating composition as defined by claim 15, further comprising a zinc or tin catalyst.

17. The coating composition as defined by claim 15, further comprising a polyol powder.

18. The coating composition as defined by claim 15, further comprising a polyamide powder.

19. The coating composition as defined by claim 15, said at least one masked isocyanate comprising an independent population of particles.

20. A process for the preparation of the masked isocyanate as defined by claim 1, comprising reacting said hydroxylated aromatic compound with said isocyanate.

21. The process as defined by claim 19, further comprising precipitating the masked isocyanate from the medium of reaction by addition of an apolar solvent thereto.

22. The process as defined by claim 21, said apolar solvent comprising an about C₄ to C₂₀ alkane.

23. A substrate coated with the composition as defined by claim 15.

24. The coating composition as defined by claim 15, comprising a paint powder.

25. The coated substrate as defined by claim 23, said at least one masked isocyanate being crosslinked within the coating therefor.
